(19) United States
(12) Patent Application Publication
(10) Pub. No.: US 2006/0097192 A1
(43) Pub. Date: May 11, 2006

(54) Method and appartus for removing target material from a substrate

(76) Inventor: Christopher Davies, Carmarthenshire (GB)

Correspondence Address: GORDON & JACOBSON, P.C., 60 LONG RIDGE ROAD, SUITE 407, STAMFORD, CT, 06902, US

(21) Appl. No.: 10/522,431

(22) PCT Filed: Jul. 18, 2003
(86) PCT No.: PCT/GB03/03248

§ 371 Date: Sep. 28, 2005

(30) Foreign Application Priority Data
Jul. 20, 2002 (GB) 0216949.8

(51) Int. Cl. A61N 5/00

(52) U.S. Cl. 250/492.3

(57) ABSTRACT

A method for removing target material from a substrate, the method comprising directing a supply of particulate material toward a target zone of target material present on the substrate and directing radiant optical energy toward the target zone, the radiant optical energy interacting with the target material and the particulate material promoting removal of target material from the substrate.

The present invention relates to a method and apparatus for removing a target material from a substrate.

In the context of the invention the terms target material and substrate should be interpreted broadly, as covering removal of a variety of coatings, coverings or marks on a variety of surfaces. Such coating, coverings or marks may be organic or inorganic materials and specifically include paints, or other materials present on substrates such as masonry, concrete, metallic or textile substrates. The invention is particularly directed to amelioration of graffiti or other substrate scarring (such as verdigris or rust) in non-sterile environments such as outdoors or in public areas. The invention covers surface treatments where the marking or coating is not completely removed but at least the substrate appearance is rejuvenated or improved.

Prior art techniques for removing material from substrates using radiant energy are known from, for example U.S. Pat. No. 6,195,505, U.S. Pat. No. 5,789,755 and U.S. Pat. No. 5,328,517.

An improved technique has now been devised.

According to a first aspect, the present invention provides a method for removing target material from a substrate, the method comprising directing a supply of particulate material toward a target zone of the substrate and directing radiant optical energy toward the target zone, the radiant optical energy interacting with the target material and the particulate material promoting removal of target material from the substrate.

It is preferred that the radiant optical energy is light energy, preferably including wavelengths in the visible range of the spectrum. The light energy may be limited to wavelengths in the visible range of the spectrum. Preferably the light energy is broadband light energy not limited to a single wavelength or narrow wavelength band.

The interaction between the radiant optical energy and the particulate material is beneficially a thermal interaction.

Beneficially, the interaction between the radiant optical energy and the target material is a thermal interaction, preferably effecting ablation or pyrolysis of the target material.

It is preferred that the interaction between the radiant optical energy and the particulate material results in a blast or shock acting at the target zone, preferably a pressure or gas blast or shock in the region of the target zone.

The interaction between the radiant optical energy and the particulate material beneficially results in the evolution of a gas having properties providing a physical or chemical interaction with material at the target zone. Such a physical interaction may be the pressure blast effect referred to above. The interaction between the radiant optical energy and the particulate material is beneficially a sublimation interaction, beneficially in which Carbon dioxide is evolved.

It is preferred that the particulate material is a material in solid state at ambient temperature. Beneficially the particulate material comprises bicarbonate of soda in particulate form such as in granular or pellet form. Advantageously the particulate material is directed across the target zone in a direction transverse to the direction of the directed radiant optical energy. The particulate material is preferably delivered entrained in a transport gas, the transport gas preferably being pressurised air.

The radiant optical energy is desirably delivered as a pulse of optical energy (preferably as a series of pulses).

It is preferred that the particulate material is directed to the target zone at times when the radiant optical energy is also directed to the target zone (i.e. contemporaneously). It is preferred that the particulate material is also directed to the target zone when radiant optical energy is not directed to the target zone, preferably including at times subsequent to delivery of radiant optical energy to the target zone.

The radiant optical energy is preferably delivered by a flashlamp delivery system, beneficially wherein the radiant optical energy is delivered in pulse form and/or the energy density of the energy at the target zone is substantially in the range 5 J/cm²-150 J/cm².

Beneficially the particulate material and the radiant optical energy is delivered via a combined delivery unit, which is desirably portable and/or hand held manipulatable.

According to one embodiment, the invention provides a method of removing graffiti or other unwanted material from an architectural or vehicle surface, the method comprising directing a supply of particulate material toward a target zone of the substrate, the particulate material being in solid phase at ambient temperature, and directing radiant optical energy toward the target zone, the radiant optical energy:
i) interacting with the target material in a thermal interaction resulting in ablation or pyrolysation of at least some of the target material; and,
ii) interacting with the particulate material in a sublimation reaction evolving a gas having a blast effect at the target zone.

According to a further aspect, the invention provides apparatus for removing target material from a substrate, the apparatus comprising:
a particulate supply arrangement configured to direct a supply of particulate material toward a target zone of the substrate; and,
a radiant optical energy delivery system configured to direct radiant optical energy toward the target zone;
   the radiant optical energy interacting with the target material and the particulate material promoting removal of target material from the substrate.

It is preferred that the radiant optical energy delivery system comprises flashlamp system, preferably arranged to deliver non-coherent light including wavelengths in the visible range of the spectrum.

The apparatus is beneficially controlled to limit the pulse rate and/or duration of a light pulse event.

The optical energy delivery system preferably includes a hand-held light delivery unit arranged to be positioned relative to the target zone manually by user.

The apparatus preferably further includes an exhaust arrangement facilitating removal of soot/pyrolysed material and the particulate material.

The apparatus preferably includes means to adjust and/or limit the pulse repetition rate of successive optical pulse event and/or the duration of an optical pulse event, and/or the intensity of the optical energy delivered; and/or the spectrum or spectrum range of the radiant optical energy.

Beneficially the optical energy delivery system includes a manually actuatable trigger for initiating a light pulse when the delivery means is positioned to the users satisfaction.

The invention will now be further described in a specific embodiment by way of example only with reference to the accompanying drawings in which:

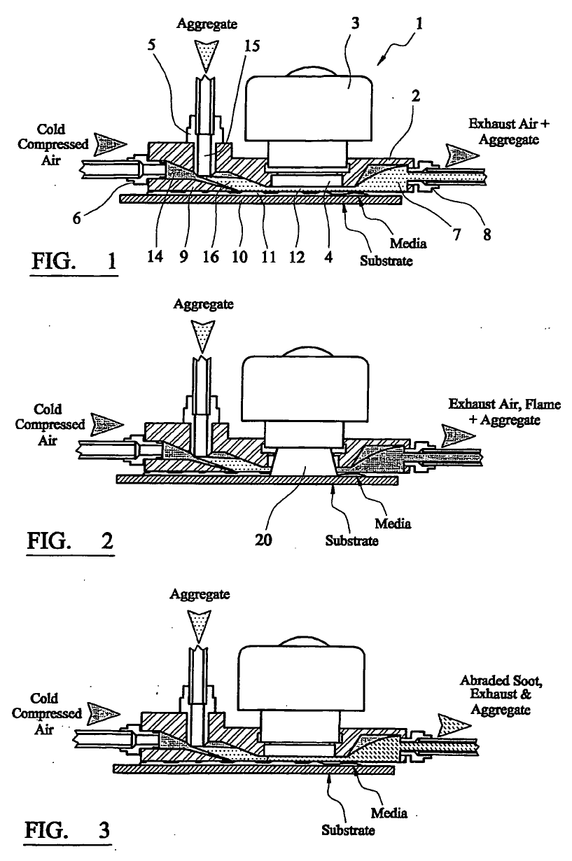

FIG. 1 is a part-sectional view of apparatus for use according to the invention in the first stage of operation;

FIG. 2 is a part-sectional view of the apparatus of FIG. 1 in a second stage of operation; and

FIG. 3 is a part-sectional view of the apparatus of FIGS. 1 and 2 in a third stage of operation.

Referring to the drawings, the apparatus 1 comprises a portable and manually manipulatable unit 1 comprising a support housing 2 for an electrical gas discharge flashlamp unit 3. Flashlamp unit 3 is mounted through a rear wall of housing 2 and has an optical output window 4 presenting into a cavity 12 in the forward face of housing 2. The unit 1 has port connections 5, 6 leading to a flow path network across the housing 2. Connection 5 is for connection to a particulate aggregate supply (typically a supply of bicarbonate of soda pellets or granules). Connection 6 is for connection to a source of compressed air.

The flow path network is defined within and at the forward surface of the support housing 2, the network comprising conduits 14, 15 leading to a common inclined wedge space 16 which connects with the cavity 12. The network directs compressed air passing via port connection 6, to transport particulate aggregate material passing via port connection 5 across cavity 12 adjacently in front of the light output window 4 of the flashlamp unit 3. The cavity 12 therefore defines a ‘target zone’ across which the particulate aggregate material is pneumatically conveyed and which is also targeted by the output window 4 of the flashlamp unit 3.

The flow path network in housing 2 is provided with an exhaust plenum 7 downstream of cavity 12 and connecting with exhaust output connection 8 for removal of exhaust air, aggregate and other materials, such as pyrolysation products (as will be described in detail later).

Forward surface portions 9 of the housing 2 are provided to ensure that the light output window 4 of flashlamp unit 3 is spaced (by the depth of cavity 12) from the substrate 10 from which a target covering material 11 is to be removed, for optimum operation.

The arrangement is particularly suited for use in removing graffiti/paint/organic material coverings, coatings or markings from substrates such as brick, metal, or the like. The general operation of the arrangement will be described hereinafter.

In the situation shown in FIG. 1, particulate bicarbonate of soda (or other suitable particulate aggregate material) is metered via port connection 5 into the cold compressed air stream passing into the flow network of housing 2 via port connection 6. At this point the flashlamp 3 is not active and the particulate aggregate has an abrasive action on the target covering material 11 present on substrate 10 causing loosely adhering target covering material 11 to break away (either inherently or following an earlier light pulse of the flashlamp at an adjacent or the same zone). If the target covering material 11 is soft in consistency, some of the particulate aggregate material (bicarbonate of soda particles) may become embedded in the target covering material 11. The compressed air, particulate aggregate material and any abraded target covering material 11 passes into the exhaust of the system via connection 8.

Referring to FIG. 2, the flashlamp unit 3 is next pulsed to produce a flash pulse 20 of radiant optical energy (light) whilst the compressed air and particulate aggregate material stream continues to pass in front of the output window 4 via cavity 12. This causes a rapid heating of coating 11 and thermal decomposition/pyrolysation thereof. Simultaneously, the solid particulate aggregate is heated and rapidly undergoes a sublimation reaction causing rapid evolution of gas at the cavity zone 12 between the output window 4 and substrate 10. This produces a pressure blast effect increasing the pressure in the cavity zone 12 between the window 4 and the substrate 10 which also aids in the exhaust of material via exhaust port 8. A variety of aggregates have been used in proving the present invention. Those aggregates which are in solid form at ambient temperature but rapidly decompose to evolve a gas on heating (sublimate) have been found to achieve best results. An example of a material which has been found to be particularly suited for this purpose is bicarbonate of soda. Such material has been repeatedly found to achieve higher levels of covering media 11 removal and lower levels of residual soot for exhaust. When the flashlamp unit 3 is pulsed the bicarbonate of soda undergoes rapid thermal decomposition producing carbon dioxide gas and water vapour momentarily increasing the pressures under the support housing 2 and providing some cooling for the substrate. The pressures generated by this interaction often causes the rapid ejection of soot, flame and unused aggregate via the exhaust connection 8. The phenomenon reported is believed also to help to control the oxidisation of the coating 11 and provide protection for the exposed substrate whilst enhancing the action of the transport compressed air stream in soot removal. The pressure blast also aids in loosening marking material not ablated/pyrolysed by the light flash. Hot vapour and combustion by-products are carried away from the cavity zone 12 adjacent the flashlamp window 4 by the transport stream of compressed air.

Following pulsing of the lamp, the arrangement operates in the state of operation shown in FIG. 3. The compressed air continues to transport the particulate solid aggregate through the flow network via cavity zone 12 past window 4, but the sublimation phase change of the particulate aggregate does not occur because the light pulse has died away. This enables the particulate aggregate to exhaust in solid form and aids in removing the residual soot (comprising the pyrolysed remains of coating 11) from the substrate 10. It has been found that the soot effectively binds to the particulate aggregate particles exhausting via the exhaust connection 8. This has environmental benefits in disposal of the waste products from the process.

Particularly on thick films of coatings such as paint, the action of the flashlamp sometimes causes a softening of the media, allowing the particulate aggregate crystals to become embedded in the coating. On sublimation decomposition under the rapid heating effect of the flashlamp 3, the embedded aggregate particulate acts to further disrupt the integrity of the coating 11 upon thermal decomposition under the influence of the next light flash rupturing from within the thickness of the coating. This causes pronounced disruption and effective removal of the coating. The flow of the aggregate in the transport air stream is effectively constant whilst the flashlamp unit 3 operates in a pulsed regime. The fact that the particulate material is in solid phase at ambient temperature ensures that a particulate not interacted with by the light energy from the flashlamp unit 3 enters the exhaust system (via connection 8) in solid particulate form.

The output of the flashlamp unit 3 is non-coherent and non-collimated which results in rapid attenuation of light intensity with distance from the output window 4, such that at a distance of, for example, 10-20 cm from the output window 4 the light intensity is of such a low level that it would not damage the skin of a user. However at a distance of up to 5 cm or so, the light intensity is at a sufficient level to effect the required ablation, thermal pyrolysation or other thermal or physical interaction with the surface sufficient to cause a rejuvenated appearance at the substrate 10 surface by removing sufficient target coating material 11 from the surface.

Beneficially, the light energy delivered during a pulse event of the flashlamp unit 3 will provide energy density at the surface substantially at or in the range 5-150 joules/cm².

Typically the flashlamp unit 3 includes one or more flashtubes and a reflector to direct the light pulse through window 4. The flashlamp unit 3 may be provided at the end of a flexible umbilical line connecting to a base unit housing a power supply and/or a control unit.

Power supply unit 10 for the apparatus includes a pulse forming network including a capacitor. The voltage dc output is used to charge the capacitor for storage of electrical energy. The capacitor remains charged until an operator or user is ready to use the apparatus. When the operator triggers the optical output, the energy stored in the capacitor is delivered to the flashtubes through a suitable high voltage switch. The electrical energy is converted by the flashtube into optical (light) energy, the duration and intensity of the optical light pulse event being determined by the amount of energy stored in the capacitor and the rate of discharge. The flashtubes of the unit 3 are typically selected to deliver light energy across a wide range of the visible spectrum. Typically, output spectrum or spectrum range is controlled and variable dependent upon end user requirements such as paint or substrate colour.

An embodiment of the present invention has been described above by way of example only. It will be apparent to persons skilled in the art that modifications and variations can be made without departing from the scope and spirit of the invention.

1. A method for removing target material from a substrate, the method comprising directing a supply of particulate material toward a target zone of target material present on the substrate and directing radiant optical energy toward the target zone, the radiant optical energy interacting with the target material and the particulate material to thereby promote removal of target material from the substrate.

2. A method according to claim 1, wherein the radiant optical energy is selected from the group of:
i) light energy;
ii) light energy that includes wavelengths in the visible range of the spectrum; and
iii) light energy that is limited to wavelengths in the visible range of the spectrum.

3-4. (canceled)

5. A method according to claim 1, wherein the interaction between the radiant optical energy and the particulate material is at least one of:
i) a thermal interaction;
ii) an interaction that results in a blast or shock medium acting at the target zone;
iii) an interaction that results in the evolution of gas having properties providing a physical or chemical interaction with material at the target zone;
iv) a sublimation interaction; and
v) an interaction that produces carbon dioxide.

6. A method according to claim 1, wherein the interaction between the radiant optical energy and the target material is at least one of a thermal interaction and an interaction effecting ablation or pyrolysis of the target material.

7-11. (canceled)

12. A method according to claim 1, wherein the particulate material is a material in solid state at ambient temperature.

13. A method according to claim 1, wherein the radiant optical energy is delivered as one of a pulse of optical energy and a series of pulses.

14. (canceled)

15. A method according to claim 1, wherein the particulate material is directed across the target zone in a direction transverse to the direction of the directed radiant optical energy.

16. A method according to claim 1, wherein the particulate material is directed to the target zone during at least one of:
i) times when the radiant optical energy is also directed to the target zone; and
ii) times when radiant optical energy is not directed to the target zone.

17. (canceled)

18. A method according to claim 1, wherein the particulate material comprises bicarbonate of soda in particulate form.

19. A method according to claim 1, wherein the particulate material is delivered entrained in a transport gas.

20. A method according to claim 19, wherein the transport gas is pressurised air.

21. A method according to any preceding claim 1, wherein the radiant optical energy is delivered by a flashlamp delivery system.

22. A method according to claim 1, wherein the radiant optical energy is delivered in pulse form, the energy density of the energy at the target zone being substantially in the range between 5 J/cm² and 150 J/cm².

23. A method according to claim 1, wherein the spectrum of the radiant optical energy is variable in a controlled manner.

24. A method according to claim 1, wherein the particulate material and the radiant optical energy is delivered via a combined delivery unit that is portable and/or hand held manipulatable.

25. (canceled)

26. A method of removing graffiti or other unwanted material from an architectural or vehicle surface, the method comprising:
directing a supply of particulate material toward a target zone of the substrate, the particulate material being in solid phase at ambient temperature; and
directing radiant optical energy toward the target zone, the radiant optical energy:
i) interacting with the target material in a thermal interaction resulting in ablation or pyrolysation of at least some of the target material, and
ii) interacting with the particulate material in a sublimation reaction evolving a gas having a blast effect at the target zone.

27. Apparatus for removing target material from a substrate, the apparatus comprising:
a particulate supply arrangement configured to direct a supply of particulate material toward a target zone of the substrate; and
a radiant optical energy delivery system configured to direct radiant optical energy toward the target zone, the radiant optical energy interacting with the target material and the particulate material to thereby promote removal of target material from the substrate.

28. Apparatus according to claim 27, wherein the radiant optical energy delivery system comprises a flashlamp system.

29. Apparatus according to claim 27, wherein the apparatus is controlled to limit at least one of the pulse rate and the duration of a light pulse event.

30. Apparatus according to claim 27, wherein the optical energy delivery system includes a hand-held light delivery unit arranged to be positioned relative to the target zone manually by user.

31. Apparatus according to claim 27, further including an exhaust arrangement facilitating removal of soot/pyrolysed material and the particulate material.

32. Apparatus according to claim 27, wherein the apparatus is controllable to deliver the light energy in the form of a pulse of light.

33. Apparatus according to claim 32, wherein the apparatus includes means to adjust at least one:
the pulse repetition rate of successive light pulses;
the duration of said pulse of light;
the intensity of said pulse of light; and
the spectrum range of said pulse of light.

34. Apparatus according to claim 27, wherein the optical energy delivery system includes a manually actuatable trigger for initiating a light pulse when the delivery means is positioned to the users satisfaction.
